SEQUENCE LISTING (38 sequences)

SEQ ID NO: 1 - DNA, 31 bp, Artificial Sequence
  Forward primer for cloning SCP17
ccaagcttggaagagctcttctctggctgtg

SEQ ID NO: 2 - DNA, 29 bp, Artificial Sequence
  Reverse primer for cloning SCP17
gacctaggtctctttgatcaaaagttttt

SEQ ID NO: 3 - DNA, 28 bp, Artificial Sequence
  Forward primer for cloning ACP5
ccaagcttgcatactctctcgtgaactc

SEQ ID NO: 4 - DNA, 26 bp, Artificial Sequence
  Reverse primer for cloning ACP5
gacctaggtatcgatctgatcgagag

SEQ ID NO: 5 - DNA, 20 bp, Artificial Sequence
  Forward primer for cloning LEC1
gatgccaaagggagcagaga

SEQ ID NO: 6 - DNA, 20 bp, Artificial Sequence
  Reverse primer for cloning LEC1
ccccttgcatcaccctcaaa

SEQ ID NO: 7 - DNA, 41 bp, Artificial Sequence
  Forward primer for cloning SpecR
ccgattttgaaaccgcggcatgatatatctcccaatttgtg

SEQ ID NO: 8 - DNA, 38 bp, Artificial Sequence
  Reverse primer for cloning SpecR
ctgcctgtgatcaccgcggtaagcctcgttcggttcgt

SEQ ID NO: 9 - DNA, 48 bp, Artificial Sequence
  Forward primer for cloning BastaR
ctcggtaccaagcttggggcgcgccgatacatgagaattaagggagtc

SEQ ID NO: 10 - DNA, 42 bp, Artificial Sequence
  Reverse primer for cloning BastaR
ctgaattaacgccgaattaatgagcttgcatgccggtcgatc

SEQ ID NO: 11 - DNA, 25 bp, Artificial Sequence
  Forward primer for SAND
aactctatgcagcatttgatccact

SEQ ID NO: 12 - DNA, 24 bp, Artificial Sequence
  Reverse primer for SAND
tgattgcatatctttatcgccatc

SEQ ID NO: 13 - DNA, 20 bp, Artificial Sequence
  Forward primer for PP2AA3
taacgtggccaaaatgatgc

SEQ ID NO: 14 - DNA, 20 bp, Artificial Sequence
  Reverse primer for PP2AA3
gttctccacaaccgcttggt

SEQ ID NO: 15 - DNA, 22 bp, Artificial Sequence
  Primer for ER332
gagctgaagtggcttccatgac

SEQ ID NO: 16 - DNA, 21 bp, Artificial Sequence
  Primer for ER333
ggtccgacatacccatgatcc

SEQ ID NO: 17 - DNA, 21 bp, Artificial Sequence
  Forward primer for AtACC1
agtgagaatgcataggttggg

SEQ ID NO: 18 - DNA, 21 bp, Artificial Sequence
  Reverse primer for AtACC1
ctcggtatatgtggacagtgc

SEQ ID NO: 19 - DNA, 17 bp, Artificial Sequence
  Forward primer for AtBCCP2
gacccggtgaaccccct

SEQ ID NO: 20 - DNA, 23 bp, Artificial Sequence
  Reverse primer for AtBCCP2
gtcaacgctgactggttttccat

SEQ ID NO: 21 - DNA, 27 bp, Artificial Sequence
  Forward primer for AtPDHE1C
atgtgtgctcaaatgtattaccgaggc

SEQ ID NO: 22 - DNA, 19 bp, Artificial Sequence
  Reverse primer for AtPDHE1C
acctttgctgagggcatgg

SEQ ID NO: 23 - DNA, 19 bp, Artificial Sequence
  Forward primer for AtSUS2
gcgggaagcaagaacaatg

SEQ ID NO: 24 - DNA, 23 bp, Artificial Sequence
  Reverse primer for AtSUS2
gaacaactcggtaaagaccaggc

SEQ ID NO: 25 - DNA, 24 bp, Artificial Sequence
  Forward primer for CsActin
acaatttcccgctctgctgttgtg

SEQ ID NO: 26 - DNA, 24 bp, Artificial Sequence
  Reverse primer for CsActin
agggtttctctcttccacatgcca

SEQ ID NO: 27 - DNA, 21 bp, Artificial Sequence
  Forward primer for CsTubulin
gggctaagggacattacactg

SEQ ID NO: 28 - DNA, 21 bp, Artificial Sequence
  Reverse primer for CsTubulin
gtgttcccataccagatccag

SEQ ID NO: 29 - DNA, 22 bp, Artificial Sequence
  Forward primer for CsEF1a
ggtaaggagattgagaaggagc

SEQ ID NO: 30 - DNA, 20 bp, Artificial Sequence
  Reverse primer for CsEF1a
cacagcaaaacgtcccaatg

SEQ ID NO: 31 - DNA, 22 bp, Artificial Sequence
  Forward primer for CsACC1
ctaagccctgaagactacgaac

SEQ ID NO: 32 - DNA, 20 bp, Artificial Sequence
  Reverse primer for CsACC1
attacccaccttgtttcccc

SEQ ID NO: 33 - DNA, 20 bp, Artificial Sequence
  Forward primer for CsBCCP2
acacagtggcatctcctttc

SEQ ID NO: 34 - DNA, 21 bp, Artificial Sequence
  Reverse primer for CsBCCP2
ggttttggcttttccgttcag

SEQ ID NO: 35 - DNA, 20 bp, Artificial Sequence
  Forward primer for CsPDHE1T
tggagaataacttgtgggcg

SEQ ID NO: 36 - DNA, 21 bp, Artificial Sequence
  Reverse primer for CsPDHE1T
accttcaacacatccataccg

SEQ ID NO: 37 - DNA, 21 bp, Artificial Sequence
  Forward primer for CsSUS2
cgtttgctacttgtcatggtg

SEQ ID NO: 38 - DNA, 21 bp, Artificial Sequence
  Reverse primer for CsSUS2
ttacccagtgattcggattgg

(19) United States
(12) Patent Application Publication
(10) Pub. No.: US 2019/0292554 A1
(43) Pub. Date: Sep. 26, 2019

(54) Increased production of storage compounds in seeds

(71) Applicant: The Regents of the University of California, Oakland, CA (US)

(72) Inventors: Henrik Vibe Scheller, Millbrae, CA (US); Linan Xie, Harbin (CN); Yerong Zhu, Nankai (CN); Dominique Loque, Vernier (CH)

(73) Assignee: The Regents of the University of California, Oakland, CA (US)

(21) Appl. No.: 16/279,466

(22) Filed: Feb. 19, 2019

Related U.S. Application Data
(60) Provisional application No. 62/631,617, filed on Feb. 16, 2018.

(51) Int. Cl. C12N 15/82; C12N 15/52

(52) U.S. Cl. CPC C12N 15/8234; C12Y 203/0102; C12N 15/52

(57) ABSTRACT

The present invention provides for a method of engineering a plant having an increased content of a target compound in the plant's seed, the method comprising introducing into the plant a first nucleic acid construct that encodes a seed-specific promoter operatively linked to a transcription factor wherein expression of the transcription factor increases the production of the target compound. A genetically modified plant cell engineered by the method.

### CROSS-REFERENCE TO RELATED APPLICATIONS

This application claims priority to U.S. Provisional Patent Application Ser. No. 62/631,617, filed on Feb. 16, 2018, which is hereby incorporated by reference.

### STATEMENT OF GOVERNMENTAL SUPPORT

The invention was made with government support under Contract Nos. DE-AC02-05CH11231 awarded by the U.S. Department of Energy. The government has certain rights in the invention.

### FIELD OF THE INVENTION

The present invention is in the field of plant gene expression.

### BACKGROUND OF THE INVENTION

Many plants accumulate oils—triacylglycerols (TAGs)—in the seeds, and such seed oils have many important uses. The global production of vegetable oils is more than 180 million tons per year, with soybean, palm seed and rapeseed oil accounting for almost 80% (webpage for: fas.usda.gov/data/oilseeds-world-markets-and-trade). An increasing fraction of vegetable oils are used for production of biodiesel. Biodiesel is an excellent fuel, but compared to lignocellulosic biofuels the yield per hectare is still low. To meet the growing demand for vegetable oils both for biodiesel and other uses there is a need to improve the yield from oil crops.

TAGs are produced in biosynthetic pathways that are generally well understood. A biotechnological approach to increase oil production in seeds is to increase the expression of biosynthetic enzymes that are limiting and represent bottlenecks in the metabolic pathways and multiple studies have taken that approach [1-4]. However, given that there are many enzymes and cofactors involved and not only one bottleneck such an approach is often not the most efficient. An alternative approach is to overexpress transcription factors that control entire pathways. Many biosynthetic pathways are controlled by master regulators, which are transcription factors that control the expression of other transcription factors. Such master regulators are ideal targets for engineering of plants with increased activity in a desired pathway since ideally only one gene needs to be upregulated to control the expression of multiple genes located downstream of the master regulator encoded by this gene. In addition, by targeting master regulatory transcription factors a whole pathway can be upregulated even if not all the enzymes, cofactors, transporters and lower-level transcription factors are known. In TAG biosynthesis, several high-level transcription factors have been identified including WRI1 (WRINKLED 1) and LEC1 (LEAFY COTYLEDON 1). LEC1 works upstream of WRI1 but both can be considered master regulators for TAG production in seeds. Several groups have tried to overexpress these transcription factors and obtained increased oil production [5-10]. However, in most cases these studies have made use of strong constitutive promoters, which cause the target genes to be expressed in many different tissues and lead to adverse effects on growth or development. Therefore, it is desirable to overexpress the master regulator only in the target tissues or cell types. One approach to tissue-specific expression is to drive the master regulator with a promoter of a downstream induced gene. In this approach, which we have designated an artificial positive feedback loop’ the target master regulator will induce its own expression after it is first induced by endogenous transcription factors, and in principle the master regulator can be expressed to very high levels but only in the cell types where it was expressed in the first place. We have shown that this approach is highly efficient in engineering plants to produce fiber cells with high density of secondary cell walls [11, 12]. We have also shown that the same principle can be used to produce plants that accumulate high amounts of leaf wax on leaf surfaces [13]. In both these cases the engineering worked significantly better than what had previously been achieved with strong constitutive promoters, which had led to adverse effects on growth. Given the reported experience with increasing oil production by overexpression of master regulators we hypothesized that the artificial positive feedback approach could give good results. Indeed, a recent report by van Erp and coworkers used a construct where WRI1 was driven by a promoter from a sucrose synthase gene that is a known downstream induced gene of WRI1 [14]. The resulting plants had about 10% higher seed oil content than control plants and did not exhibit poor growth and development. We hypothesized that using LEC1 which works upstream of WRI1 and controls more genes required for oil seed development and TAG biosynthesis might give even better results. Overexpression of a maize LEC1 homolog driven by OLEOSIN (OLE) or EARLY EMBRYO PROTEIN (EAP1) promoters has been reported, but while substantial increases in seed oil were observed, the plants showed poor seed germination and leaf growth [10].

### SUMMARY OF THE INVENTION

The present invention provides for a method of engineering a plant having an increased content of a target compound in the plant's seed, the method comprising introducing into the plant a first nucleic acid construct that encodes a seed-specific promoter operatively linked to a transcription factor wherein expression of the transcription factor increases the production of the target compound. The term “seed-specific promoter” means a promoter than expresses at a level higher in cells that lead to the formation of a seed than in other plant cells. It encompasses promoters that express highly in cells that lead to the formation of a seed but not at all or only a little in other plant cells.

In some embodiments, the seed-specific promoter is serine carboxypeptidase-like (SCPL17) promoter or Acyl Carrier Protein (ACP5) promoter. In some embodiments the promoter is a SUS2 (Sucrose Synthase 2) promoter, a PER (Peroxidase superfamily protein) promoter, a PER1 (CYSTEINE PEROXIREDOXIN 1) promoter, a BZIP67 (Basic Leucine Zipper Transcription Factor 67) promoter, or a KCS18 (3-Ketoacyl-CoA Synthase 18) promoter. In some embodiments, the SCPL17 promoter is the Arabidopsis thaliana SCPL17 promoter. In some embodiments, the ACP5 promoter is the Arabidopsis thaliana ACP5 promoter. In some embodiments, the SUS2 promoter is the Arabidopsis thaliana SUS2 (At5g49190, Sucrose Synthase 2) promoter. In some embodiments, the PER promoter is the Arabidopsis thaliana PER (At4g25980, Peroxidase superfamily protein) promoter. In some embodiments, the PER1 promoter is the Arabidopsis thaliana PER1 (At1g48130,1-CYSTEINE PEROXIREDOXIN 1) promoter. In some embodiments, the BZIP67 promoter is the Arabidopsis thaliana BZIP67 (AT3G44460, Basic Leucine Zipper Transcription Factor 67) promoter. In some embodiments, the KCS18 promoter is the Arabidopsis thaliana KCS18 (AT4G34520, 3-Ketoacyl-CoA Synthase 18) promoter. In some embodiments, the seed-specific promoter is the hypothetical protein (At3g63040, LOCATED IN endomembrane system protein) promoter.

In some embodiments, the target compound is a lipid or fatty acid.

In some embodiments, when the target compound is a lipid or fatty acid, the transcription factor is LEAFY COTYLEDON1 (LEC1) or WRINKLED 1 (WRI1). In some embodiments, the LEC1 is Zea mays LEC1. In some embodiments, the WRI1 is Zea mays WRI1 or Arabidopsis thaliana (WRI1). The use of LEC1 in the invention provides a higher yield in the production of the target compound than the use of WRI1.

In some embodiments, the method further comprises introducing a second nucleic acid construct that encodes a seed-specific promoter operatively linked to a biosynthetic enzyme involved in the biosynthesis of the target compound. In some embodiments, the first nucleic acid construct further encodes a seed-specific promoter operatively linked to a biosynthetic enzyme involved in the biosynthesis of the target compound.

In some embodiments, when the target compound is a lipid or fatty acid, the biosynthetic enzyme is diacylglycerol O-acyltransferase 1 (DGAT1).

In some embodiments, the method further comprises engineering the plant such that an endogenous enzyme, or an enzyme native to the plant, that decreases synthesis of, or catalyzes into another compound, the target compound is downregulated. An example is the engineering of the plant to comprise a construct which expresses an RNAi specific for suppressing or decreasing the expression of the enzyme.

In some embodiments, when the target compound is a lipid or fatty acid, the enzyme is lipase sugar-dependent 1 (SDP1).

In some embodiments, the target compound is a protein, starch, or a storage polysaccharide, such as beta-glucan or mannan.

In some embodiments, the plant is selected from the group consisting of Arabidopsis, Camelina, flax, poplar, eucalyptus, rice, corn, switchgrass, sorghum, millet, miscanthus, sugarcane, pine, alfalfa, wheat, soy, barley, turfgrass, tobacco, hemp, bamboo, rape, sunflower, willow, and Brachypodium.

In some embodiments, the present invention provides plants, plant cells, seeds, flowers, leave, fruit, or biomass comprising plant cells engineered to have lignin deposition that is substantially localized to the vessels of xylem tissue of the plant.

The present invention also provides for a genetically modified plant cell, comprising a first nucleic acid construct that encodes a seed-specific promoter operatively linked to a transcription factor wherein expression of the transcription factor increases the production of the target compound.

In some embodiments, the genetically modified plant cell further comprises a second nucleic acid construct that encodes a seed-specific promoter operatively linked to a biosynthetic enzyme involved in the biosynthesis of the target compound, or the first nucleic acid construct further encodes a seed-specific promoter operatively linked to a biosynthetic enzyme involved in the biosynthesis of the target compound.

In some embodiments, the plant cell is engineered such that an endogenous enzyme, or an enzyme native to the plant cell, that decreases synthesis of, or catalyzes into another compound, the target compound is downregulated.

The present invention also provides for a genetically modified plant or seed comprising a first nucleic acid construct that encodes a seed-specific promoter operatively linked to a transcription factor wherein expression of the transcription factor increases the production of the target compound.

The present invention also provides for a method of producing a target compound from a plant, comprising (a) optionally engineering a plant having an increased content of a target compound in the plant's seed using the method of claim 1, (b) growing the plant such that seeds are produced, (c) optionally harvesting the seeds produced by the plant, and (d) replanting the seeds harvested from step (c), or separating or isolating the target compound in the seeds harvested from step (c) from some, essentially all, or all of the other components of the seeds.

In some embodiments, the yield of the target compound produced by a plant or plant cell of the present invention is equal to or more than about 15%, 20%, 25%, 30%, 40%, or 50% more than the yield of a plant or plant cell that was not engineered or genetically modified as per the invention.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1A. Histochemical analysis of GUS expression under the control of pSCP17 and pACP promoters in leaves, flowers, siliques, endosperm and embryo.

FIG. 1B. T-DNA constructs designed for seed-specific overexpression of ZmLEC1. LB, left border; BAR, Basta® resistance gene; pSCP, promoter of SCP17; LEC1, LEAFY COTYLEDON 1; RB, right border.

FIG. 2. Detection of transgene by PCR analysis of genomic DNA isolated from wild type (WT), transgenic Arabidopsis plants (AtSL1, AtSL4, AtSL5) and transgenic Camelina plants (CsAL1, CsAL5, CsSL1, CsSL2) transformed with ZmLEC1 constructs. Plasmid positive controls (+) and non-template controls (−) were included.

FIG. 3A. Expression of ZmLEC1 in developing transgenic Arabidopsis seeds. Total RNA was isolated from developing seeds 6 to 9 DAF and subjected to RT-PCR analyses. The PP2AA3 gene was used as a housekeeping gene to confirm the quality and quantity of RNA.

FIG. 3B. Expression of ZmLEC1 in developing transgenic Camelina seeds. Total RNA was isolated from developing seeds 15 DAF. The CsEF1f gene was used as a housekeeping gene to confirm the quality and quantity of RNAs. WT is the wild type, AtSL are independent homozygous T3 lines expressing the pSCPL17:ZmLEC1 transgenes in Arabidopsis. CsAL and CsSL are independent homozygous T3 lines expressing the pSCPL17:ZmLEC1 and pACP5:ZmLEC1 transgenes, respectively, in Camelina.

FIG. 4A. Seed oil content of independent transgenic lines expressing ZmLEC1 in Arabidopsis. Oil content in mature seeds of wild-type plants and transformants was determined by NMR. Wild-type and transgenic plants are designated as explained in legend to FIG. 3A. Significant differences compared to wild type (Student's t-test) are indicated: **P<0.01 and ***P<0.001. Error bars indicate standard deviation, n=6.

FIG. 4B. Seed oil content of independent transgenic lines expressing ZmLEC1 in Camelina. Oil content in mature seeds of wild-type plants and transformants was determined by NMR. Wild-type and transgenic plants are designated as explained in legend to FIG. 3B. Significant differences compared to wild type (Student's t-test) are indicated: **P<0.01 and ***P<0.001. Error bars indicate standard deviation, n=6.

FIG. 5A. Effect of ZmLEC1 expression on early seedling growth rate and plant height in Arabidopsis. Wild-type and transgenic plants are designated as explained in legend to FIG. 3A. Significant differences compared to wild type (Student's t568 test) are indicated: *P<0.05 and **P<0.01. Error bars indicate standard deviation, n=6.

FIG. 5B. Effect of ZmLEC1 expression on early seedling growth rate and plant height in Camelina. Wild-type and transgenic plants are designated as explained in legend to FIG. 3B. Significant differences compared to wild type (Student's t568 test) are indicated: *P<0.05 and **P<0.01. Error bars indicate standard deviation, n=6.

FIG. 6A. Effects of ZmLEC1 expression on average seed mass per 100 seeds in Arabidopsis. Wild-type and transgenic plants are designated as explained in legend to FIG. 3A. Significant differences compared to wild type (Student's t-test) are indicated: *P<0.05. Error bars indicate standard deviation, n=6.

FIG. 6B. Effects of ZmLEC1 expression on average seed yield per plant in Arabidopsis. Wild-type and transgenic plants are designated as explained in legend to FIG. 3A. Significant differences compared to wild type (Student's t-test) are indicated: *P<0.05. Error bars indicate standard deviation, n=6.

FIG. 6C. Effects of ZmLEC1 expression on average oil yield per plant in Arabidopsis. Wild-type and transgenic plants are designated as explained in legend to FIG. 3A. Significant differences compared to wild type (Student's t-test) are indicated: *P<0.05. Error bars indicate standard deviation, n=6.

FIG. 7A. Effects of ZmLEC1 expression on average seed yield per plant in Camelina. Wild-type and transgenic plants are designated as explained in legend to FIG. 3B. No significant differences compared to wild type were found. Error bars indicate standard deviation, n=6.

FIG. 7B. Effects of ZmLEC1 expression on average oil yield per plant in Camelina. Wild-type and transgenic plants are designated as explained in legend to FIG. 3B. No significant differences compared to wild type were found. Error bars indicate standard deviation, n=6.

FIG. 8. Analysis of SUS2, P 582 DH E1α, BCCP2, ACC1 expression using quantitative reverse transcription-PCR on developing siliques of Arabidopsis. Wild-type and transgenic plants are designated as explained in legend to FIG. 3A. Significant differences compared to wild type (Student's t-test) are indicated: **P<0.01, ***P<0.001 and ****P<0.0001. Error bars indicate standard deviation, n=6.

FIG. 9. Analysis of SUS2, PDH E1α, BCCP2, ACC1 expression using quantitative reverse transcription-PCR on developing seeds of Camelina transgenic lines. Wild-type and transgenic plants are designated as explained in legend to FIG. 3B. Significant differences compared to wild type (Student's t-test) are indicated: **P<0.01, ***P<0.001 and ****P<0.0001. Error bars indicate standard deviation, n=6.

### DETAILED DESCRIPTION OF THE INVENTION

Before the invention is described in detail, it is to be understood that, unless otherwise indicated, this invention is not limited to particular sequences, expression vectors, enzymes, host microorganisms, or processes, as such may vary. It is also to be understood that the terminology used herein is for purposes of describing particular embodiments only, and is not intended to be limiting.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

As used in the specification and the appended claims, the singular forms “a,” “an,” and “the” include plural referents unless the context clearly dictates otherwise. Thus, for example, reference to an “expression vector” includes a single expression vector as well as a plurality of expression vectors, either the same (e.g., the same operon) or different; reference to “cell” includes a single cell as well as a plurality of cells; and the like.

In this specification and in the claims that follow, reference will be made to a number of terms that shall be defined to have the following meanings:

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

The term “about” refers to a value including 10% more than the stated value and 10% less than the stated value.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

The terms “polynucleotide” and “nucleic acid” are used interchangeably and refer to a single or double-stranded polymer of deoxyribonucleotide or ribonucleotide bases read from the 5′ to the 3′ end. A nucleic acid of the present invention will generally contain phosphodiester bonds, although in some cases, nucleic acid analogs may be used that may have alternate backbones, comprising, e.g., phosphoramidate, phosphorothioate, phosphorodithioate, or O-methylphophoroamidite linkages (see Eckstein, Oligonucleotides and Analogues: A Practical Approach, Oxford University Press); positive backbones; non-ionic backbones, and non-ribose backbones. Thus, nucleic acids or polynucleotides may also include modified nucleotides that permit correct read-through by a polymerase. “Polynucleotide sequence” or “nucleic acid sequence” includes both the sense and antisense strands of a nucleic acid as either individual single strands or in a duplex. As will be appreciated by those in the art, the depiction of a single strand also defines the sequence of the complementary strand; thus the sequences described herein also provide the complement of the sequence. Unless otherwise indicated, a particular nucleic acid sequence also implicitly encompasses variants thereof (e.g., degenerate codon substitutions) and complementary sequences, as well as the sequence explicitly indicated. The nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc.

The term “promoter,” as used herein, refers to a polynucleotide sequence capable of driving transcription of a DNA sequence in a cell, such as a plant cell. Thus, promoters used in the polynucleotide constructs of the invention include cis- and trans-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) gene transcription. Promoters are located 5′ to the transcribed gene, and as used herein, include the sequence 5′ from the translation start codon (i.e., including the 5′ untranslated region of the mRNA, typically comprising 100-200 bp). By convention, the promoter sequence is usually provided as the sequence on the coding strand of the gene it controls. In the context of this application, a promoter is typically referred to by the name of the gene for which it naturally regulates expression. A promoter used in an expression construct of the invention is referred to by the name of the gene. Reference to a promoter by name includes a wildtype, native promoter as well as variants of the promoter that retain the ability to induce expression. Reference to a promoter by name is not restricted to a particular species, but also encompasses a promoter from a corresponding gene in other species.

The term “heterologous” as used herein refers to a material, or nucleotide or amino acid sequence, that is found in or is linked to another material, or nucleotide or amino acid sequence, wherein the materials, or nucleotide or amino acid sequences, are foreign to each other (i.e., not found or linked together in nature, such as within the same species of organism). A polynucleotide is “heterologous” to an organism or a second polynucleotide sequence if it originates from a foreign species, or, if from the same species, is modified from its original form. For example, when a polynucleotide encoding a polypeptide sequence is said to be operably linked to a heterologous promoter, it means that the polynucleotide coding sequence encoding the polypeptide is derived from one species whereas the promoter sequence is derived from another, different species; or, if both are derived from the same species, the coding sequence is not naturally associated with the promoter (e.g., is a genetically engineered coding sequence, e.g., from a different gene in the same species, or an allele from a different ecotype or variety).

The term “operably linked” refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a DNA or RNA sequence if it stimulates or modulates the transcription of the DNA or RNA sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

The term “cassette” or construct” refers to a nucleic acid construct that, when introduced into a host cell, results in transcription and/or translation of an RNA or polypeptide, respectively. Antisense or sense constructs that are not or cannot be translated are expressly included by this definition. In the case of both expression of transgenes and suppression of endogenous genes (e.g., by antisense, RNAi, or sense suppression) one of skill will recognize that the inserted polynucleotide sequence need not be identical, but may be only substantially identical to a sequence of the gene from which it was derived.

Loque et al. (“SPATIALLY MODIFIED GENE EXPRESSION IN PLANTS”, U.S. Patent Application Pub. No. 2014/0298539), hereby incorporated by reference in its entirety, have disclosed a nucleic acid encoding a transcription factor as described in Loque et al. that regulates lipid biosynthesis and, e.g., accumulation in seed and other tissues, operably linked to a promoter from a downstream induced gene involved in lipid biosynthesis where expression of the downstream gene is induced by the transcription factor. Loque et al. also disclose: Additional examples of biosynthetic pathways that can employ an APFL include lipid biosynthetic pathways. For example, it is known that lipid biosynthesis and accumulation in seeds and other tissues occurs in specific cell types and is regulated by transcription factors such as WRI1 (WRINKLED; At3g54320), LEC1 (At1g21970), or LEC2 (At1g28300). These transcription factors can thus be used to create an AFPL to increase the accumulation of lipids in a desired tissue such as seed. Other transcription factors and appropriate promoters for use in an APFL can also be identified for other biosynthetic pathways. Lipid biosynthesis pathways are discussed, e.g., in Ohlrogge & Browse, Plant Cell 7:957, 1995; Hildebrand, et al., Plant Lipids: Biology, Utilisation and Manipulation, 67-102 (2005); and Dyer & Mullen, Seed Sci. Res. 15:255-267 (2005). An APFL using WRI1 was employed by van Erp et al, Plant Physiol. 2014; 165:30-36, where the promoter was from a sucrose synthase gene. While good results were obtained with this approach, the approach in the present invention using LEC1 promoter was substantially better leading to higher oil content. This shows that the choice of promoter-transcription factor combination is important. In general, it is best to use transcription factors that operate as far upstream as possible without resulting in adverse phenotypes. LEC1 is operating upstream of WRI1. A promoter operating too far downstream may not be controlling all the biosynthetic genes and substrate metabolic pathways that are required for high production of the target compound. An ideal master regulator should control all the relevant biosynthetic enzymes as well as enzymes required for substrate production.

It is reported herein that a number of different seed-specific promoters were tested and used to drive the expression of ZmWRI1, AtWRI1, and ZmLEC1 in Arabidopsis and Camelina. The best results were obtained with LEC1, which works upstream of WRI1. To identify the best promoters, the expression pattern using promoter-GUS constructs were investigated. Based on the GUS analysis and comparison of different lines, SCP (serine carboxypeptidase-like 17, At3g12203) was identified as a strong and very seed-specific promoter. Another good promoter identified is the promoter for ACP (acyl Carrier Protein, At5g27200), which is stronger but not as seed-specific. These promoter combinations with LEC1 worked better than previously disclosed constructs. Some embodiments of the present invention comprise the combination of a strong seed-specific promoter with LEC1. The results obtained show that the best result are obtained with LEC1, as compared to WRI1. This is probably because LEC1 works upstream of WRI1. The best results are obtained with combination of promoters that are both strong and seed-specific. It is not sufficient that the promoter works in oil biosynthesis, because expression in non-seed tissue leads to less favorable results. On the other hand, some degree of expression in non-seed tissues may still be acceptable or necessary for growth and development.

In Arabidopsis, the best results were obtained with pSCP:ZmLEC1, where one finds an increase in oil content of 21%, as compared to the reported maximum of 10% in an earlier disclosure. In Camelina, a 15% increase in seed oil is obtained with both pACP:ZmLEC1 and pSCP:ZmLEC1, as compared to the 10% reported in an earlier disclosure.

It is important to use a promoter of the appropriate strength for constructs such as those described herein. The present invention can be used together with other engineered constructs. In particular, overexpression of diacylglycerol acyl transferase (DGAT1) and downregulation of the lipase sugar-dependent 1 (SDP1) have been shown to increase the accumulation of oil when combined with overexpression of WRI1. These combinations cam also be sued with the over expression of ZmLEC1 to further increase the seed oil content. Furthermore, one can overexpress LEC1 and WRI1 together as they may not control exactly the same downstream genes. Downregulation of SDP1 can be achieved with RNAi as reported in an earlier disclosure. However, a better and more specific method would be to use tissue-specific genome engineering with CRISPR/Cas9 as described in Loque et al. (“GENERATION OF HERITABLE CHIMERIC PLANT TRAITS”, U.S. Patent Application Pub. No. 2015/0218573), hereby incorporated by reference in its entirety.

We have demonstrated the substantial increase of seed oil in Arabidopsis and Camelina. The same or similar constructs can be used in any other oil crop, such as rapeseed, soybean, flax, and the like. AFPL works for the production of seed storage compounds.

### REFERENCES CITED

[1] Vigeolas H, Waldeck P, Zank T, Geigenberger P. Increasing seed oil content in oil-seed rape (Brassica napus L.) by over-expression of a yeast glycerol-3-phosphate dehydrogenase under the control of a seed-specific promoter. Plant Biotechnol J. 2007; 5:431-441.
[2] Kelly A A, Shaw E, Powers S J, Kurup S, Eastmond P J. Suppression of the SUGAR-DEPENDENT1 triacylglycerol lipase family during seed development enhances oil yield in oilseed rape (Brassica napus L.). Plant Biotechnol J. 2013; 11:355-361.
[3] Kim M J, Yang S W, Mao H Z, Veena S P, Yin J L, Chua N H. Gene silencing of Sugar-dependent 1 (JcSDP1), encoding a patatin-domain triacylglycerol lipase, enhances seed oil accumulation in Jatropha curcas. Biotechnol Biofuels. 2014; 7:36.
[4] Jako C, Kumar A, Wei Y, Zou J, Barton D L, Giblin E M, Covello P S, Taylor D C. Seed-specific over-expression of an Arabidopsis cDNA encoding a diacylglycerol acyltransferase enhances seed oil content and seed weight. Plant Physiol 2001; 126:861-874.
[5] Lotan T, Ohto M, Yee K M, West M A, Lo R, Kwong R W, Yamagishi K, Fischer R L, Goldberg R B, Harada J J. Arabidopsis LEAFY COTYLEDON1 is sufficient to induce embryo development in vegetative cells. Cell. 1998; 93:1195-1205.
[6] Stone S L, Kwong L W, Yee K M, Pelletier J, Lepiniec L, Fischer R L, Goldberg R B, Harada J J. LEAFY COTYLEDON2 encodes a B3 domain transcription factor that induces embryo development. Proc Natl Acad Sci USA. 2001; 98:11806-11811.
[7] Cernac A, Benning C. WRINKLED1 encodes an AP2/EREB domain protein involved in the control of storage compound biosynthesis in Arabidopsis. Plant J. 2004; 40:575-585.
[8] Wang H, Guo J, Lambert K N, Lin Y. Developmental control of Arabidopsis seed oil biosynthesis. Planta. 2007; 226:773-783.
[9] Tan H, Yang X, Zhang F, Zheng X, Qu C, Mu J, Fu F, Li J, Guan R, Zhang H et al. Enhanced seed oil production in canola by conditional expression of Brassica napus LEAFY COTYLEDON1 and LEC1-LIKE in developing seeds. Plant Physiol. 2011; 156:1577-1588.
[10] Shen B, Allen W B, Zheng P, Li C, Glassman K, Ranch J, Nubel D, Tarczynski M C. Expression of ZmLEC1 and ZmWRI1 increases seed oil production in maize. Plant Physiol. 2010; 153:980-987.
[11] Yang F, Mitra P, Zhang L, Prak L, 482 Verhertbruggen Y, Kim J S, Sun L, Zheng K, Tang K, Auer M et al. Engineering secondary cell wall deposition in plants. Plant Biotechnol J. 2013; 11:325-335.
[12] Gondolf V M, Stoppel R, Ebert B, Rautengarten C, Liwanag A J, Loque D, Scheller H V. A gene stacking approach leads to engineered plants with highly increased galactan levels in Arabidopsis. BMC Plant Biol. 2014; 14:344.
[13] Loque D, Scheller, H. V. Spatially modified gene expression in plants. U S patent application PCT/US2012/023182. Published Aug. 2, 2012.
[14] van Erp H, Kelly A A, Menard G, Eastmond P J. Multigene engineering of triacylglycerol metabolism boosts seed oil content in Arabidopsis. Plant Physiol. 2014; 165:30-36.
[15] Baud S, Mendoza M S, To A, Harscoet E, Lepiniec L, Dubreucq B. WRINKLED1 specifies the regulatory action of LEAFY COTYLEDON2 towards fatty acid metabolism during seed maturation in Arabidopsis. Plant J. 2007; 50:825-838.
[16] Obayashi T, Hayashi S, Saeki M, Ohta H, Kinoshita K. ATTED-II provides coexpressed gene networks for Arabidopsis. Nucleic Acids Res. 2009; 37:D987-991.
[17] Fraser C M, Rider L W, Chapple C. An expression and bioinformatics analysis of the Arabidopsis serine carboxypeptidase-like gene family. Plant Physiol. 2005; 138:1136-1148.
[18] Winter D, Vinegar B, Nahal H, Ammar R, Wilson G V, Provart N J. An “Electronic Fluorescent Pictograph” browser for exploring and analyzing large 504 scale biological data sets. PLoS One. 2007; 2:e718.
[19] Sanjaya, Durrett T P, Weise S E, Benning 505 C. Increasing the energy density of vegetative tissues by diverting carbon from starch to oil biosynthesis in transgenic Arabidopsis. Plant Biotechnol J. 2011; 9:874-883.
[20] Grotewold E. Transcription factors for predictive plant metabolic engineering: are we there yet? Curr Opin Biotechnol. 2008; 19:138-144.
[21] Broun P. Transcription factors as tools for metabolic engineering in plants. Curr Opin Plant Biol. 2004; 7:202-209.
[22] An D, Suh M C. Overexpression of Arabidopsis WRI1 enhanced seed mass and storage oil content in Camelina sativa. Plant Biotechnology Reports. 2015; 9:137-148.
[23] Meyer K, Damude H G, Everard J D, Ripp K G, Stecca K L. Use of a seed specific promoter to drive odp1 expression in cruciferous oilseed plants to increase oil content while maintaining normal germination. U S patent application PCT/US2010/029609. Published Oct. 7, 2010.
[24] Vega-Sanchez M E, Loque D, Lao J, Catena M, Verhertbruggen Y, Herter T, Yang F, Harholt J, Ebert B, Baidoo E E et al. Engineering temporal accumulation of a low recalcitrance polysaccharide leads to increased C6 sugar content in plant cell walls. Plant Biotechnol J. 2015; 13:903-914.
[25] Earley K W, Haag J R, Pontes O, Opper K, Juehne T, Song K, Pikaard C S. Gateway-compatible vectors for plant functional genomics and proteomics. Plant J. 2006; 45:616-629.
[26] Clough S J, Bent A F. Floral dip: a simplified method for Agrobacterium mediated transformation of Arabidopsis thaliana. Plant J. 1998; 16:735-743.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

The invention having been described, the following examples are offered to illustrate the subject invention by way of illustration, not by way of limitation.

### Example 1

### A Transgene Design for Enhancing Oil Content in Arabidopsis and 2 Camelina Seeds

Background:

Increasing the oil yield is a major objective for oilseed crop improvement. Oil biosynthesis and accumulation are influenced by multiple genes involved in embryo and seed development. The LEAFY COTYLEDON1 (LEC1) is a master regulator of embryo development that also enhances the expression of genes involved in fatty acid (FA) biosynthesis. We speculated that seed oil could be increased by targeted overexpression of a master regulating transcription factor for oil biosynthesis, using a downstream promoter for a gene in the oil biosynthesis pathway. To verify the effect of such a combination on seed oil content, we made constructs with maize (Zea mays) ZmLEC1 driven by serine carboxypeptidase-like (SCPL17) and Acyl Carrier Protein (ACP5) promoter, respectively, for expression in transgenic Arabidopsis thaliana and Camelina sativa.

Results:

Agrobacterium-mediated transformation successfully generated Arabidopsis and Camelina lines that overexpressed ZmLEC1 under the control of a seed-specific promoter. This overexpression does not appear to be detrimental to seed vigor under laboratory conditions and did not cause observable abnormal growth phenotypes throughout the life cycle of the plants. Overexpression of ZmLEC1 increased the oil content in mature seeds by more than 20% in Arabidopsis and 16% in Camelina.

### Conclusion:

The findings demonstrated that the maize master regulator, ZmLEC1, driven by a downstream seed-specific promoter, can be used to increase oil production in Arabidopsis and Camelina and might be a promising target for increasing oil yield in oilseed crops.

We tested the effect of overexpressing a LEC1 ortholog from maize (ZmLEC1) using two different downstream promoters from Arabidopsis. We transformed both the model plant Arabidopsis and the oilseed crop Camelina (Camelina sativa). In both cases we achieved up to 20% increased oil yields and we did not observe any adverse effects on growth and development. These results highlight the potential application of seed-specific overexpression of LEC1 for increasing oil production in major crops.

### Results

### Selection of Promoters

The ideal promoter for the type of positive feedback we wanted to test should have the following properties: 1) be a direct or indirect target of the endogenous LEC1 and its orthologs, 2) be involved in oil biosynthesis or storage, 3) not be expressed in other tissues than those in developing seeds, 4) be relatively strong. We investigated published data regarding the downstream targets of LEC1 and WRI1 from Arabidopsis (itself a target of LEC1) [15] and identified several candidate genes: ACP5 (At5g27200, Acyl Carrier Protein 5), SUS2 (At5g49190, Sucrose Synthase 2), PER (At4g25980, Peroxidase superfamily protein), hypothetical protein (At3g63040, LOCATED IN endomembrane system protein), SCPL17 (At3g12203, Serine Carboxypeptidase-Like 17), PER1 (At1g48130,1-CYSTEINE PEROXIREDOXIN 1), BZIP67 (AT3G44460, Basic Leucine Zipper Transcription Factor 67), KCS18 (AT4G34520, 3-Ketoacyl-CoA Synthase 18). Some of these genes encode proteins that are key enzymes in the fatty acid biosynthetic pathway, which usually coexpress with transcriptional factors regulating fatty acid biosynthesis in seeds. We have used ATTED-II (atted.jp) to analyze coexpression patterns [16]. For example, ACP5 is a key protein directly involved in TAG biosynthesis and it is highly coexpressed with the WRI1 transcription factor (At3g54320) in walking130 stick seed and torpedo embryo (webpage for: atted.jp/cgibin/coexpression_viewer.cgi?loc1=832778&loc2=824599). SCPL17 is expressed almost exclusively in siliques in Arabidopsis [17].

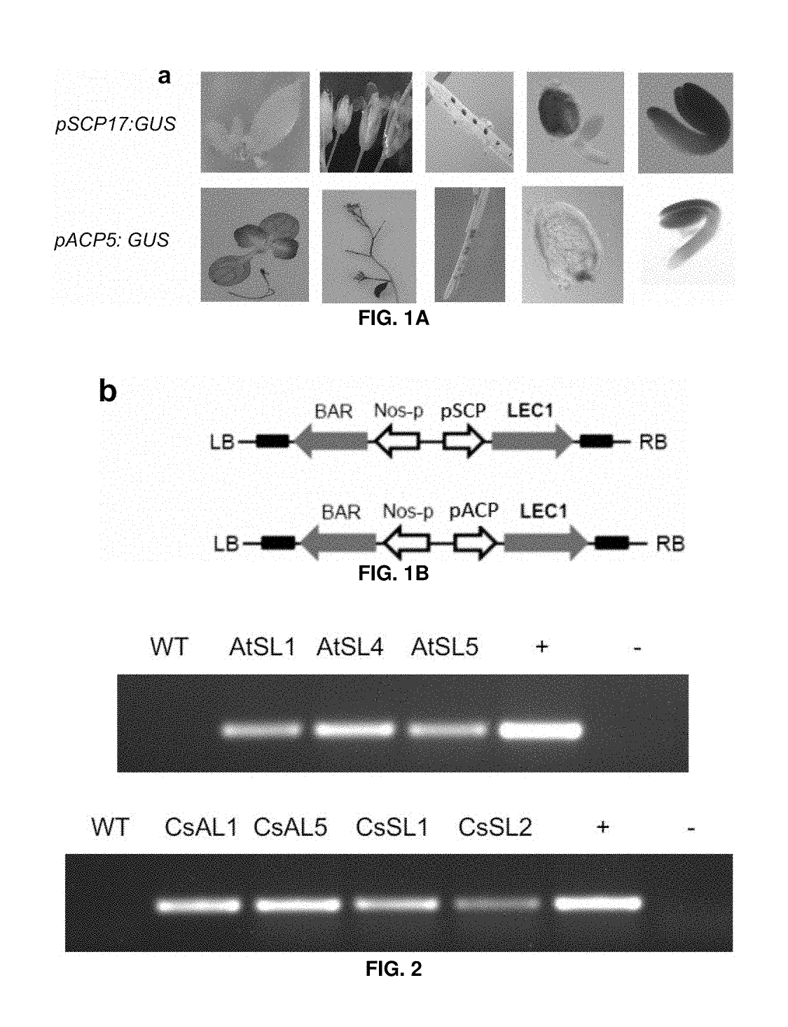

To verify tissue specific expression of these promoters, we fused them with GUS (beta134 glucuronidase) gene and transformed Arabidopsis plants. The GUS analysis showed activity with all the chosen promoters, except for that of KCS18. Plants transformed with pSCPL17:GUS showed a very specific expression restricted only to developing seeds, whereas transformation with pACP5: GUS likewise resulted in high expression in developing seeds but also showed some expression in vegetative tissues (FIG. 1A). Since the GUS staining results indicated that SCPL17 and ACP5 had the desired properties we selected these promoters to drive the 140 expression of ZmLEC1 in transgenic Arabidopsis and Camelina plants.

### Generation of Transgenic Arabidopsis Expressing ZmLEC1

After we had confirmed the expression patterns for the two promoters, they were fused individually to ZmLEC1 for overexpression in Arabidopsis and Camelina (FIG. 1B). We hypothesized that the validation of non-host derived promoters and transcription factors would increase the chance that obtained phenotype could be transferable to a large diversity of plant species while minimizing potential silencing of the transgene and endogenous genes. Likewise, the use of a protein from a distant species could minimize the risk of undesired post-translational modifications.

The binary vectors containing ZmLEC1 under the control of a seed-specific AtSCPL17 (pSCP17) or AtACP5 (pACP5) promoter were introduced into Arabidopsis and Camelina using the Agrobacterium-mediated floral dip method. Transgenic Arabidopsis and Camelina T1 seedlings were selected by hygromycin and Basta, respectively, supplemented in the medium, and resistant lines were confirmed by PCR amplification (FIG. 2).

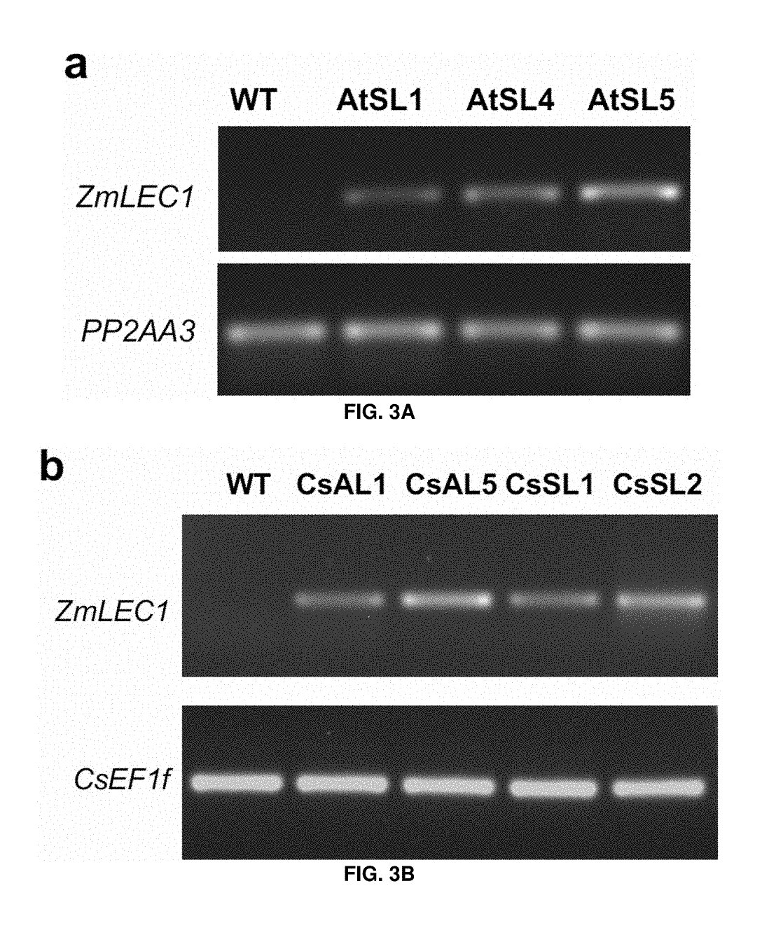

To confirm the expression of the target genes (ZmLEC1), reverse transcription-PCR (RT160 PCR) analysis was performed on developing Arabidopsis siliques containing seeds in developmental stages 6 to 9 [18] and Camelina developing seeds at 15 days after flowering (DAFs) (FIGS. 3A and 3B). ZmLEC1 transcripts were detected in all transformed lines except in the wild type. Three lines representing 163 transgenic Arabidopsis expressing pSCPL17:ZmLEC1 (AtSL1, AtSL4, AtSL5) and two lines representing Camelina expressing pSCP17:ZmLEC1(CsSL1, CsSL2) or pACP5: ZmLEC1 (CsAL1, CsAL5) were selected for further studies and shown in FIGS. 3A and 3B.

### ZmLEC1 Expression Boosts Seeds Oil Content in Arabidopsis and Camelina Seeds

Six plants from each transformed line were screened for elevated seed oil content in the T2 generation using a Mini-spec mq10 nuclear magnetic resonance (NMR) analyzer (Bruker optics Inc., Houston, Tex., USA) and low-resolution time domain NMR spectroscopy (Hobbs et al., 2004). For each construct, the transgenic lines exhibited a range in percentage seed oil content. Lines with the highest percentage oil content were then taken to produce the T3 generation. Six homozygous lines harboring a single175 insertion were identified for each construct by segregation analysis.

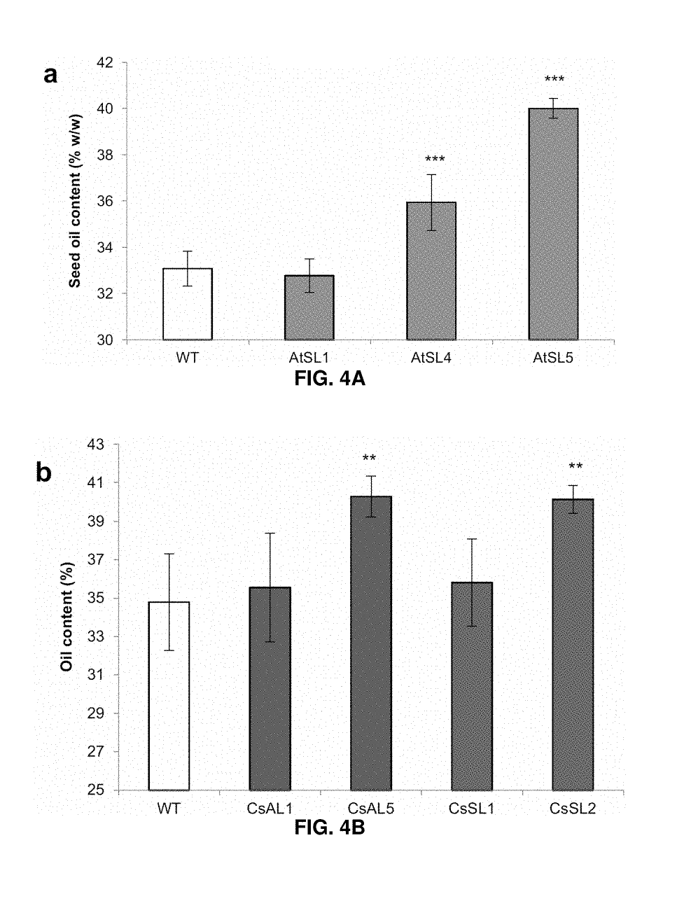

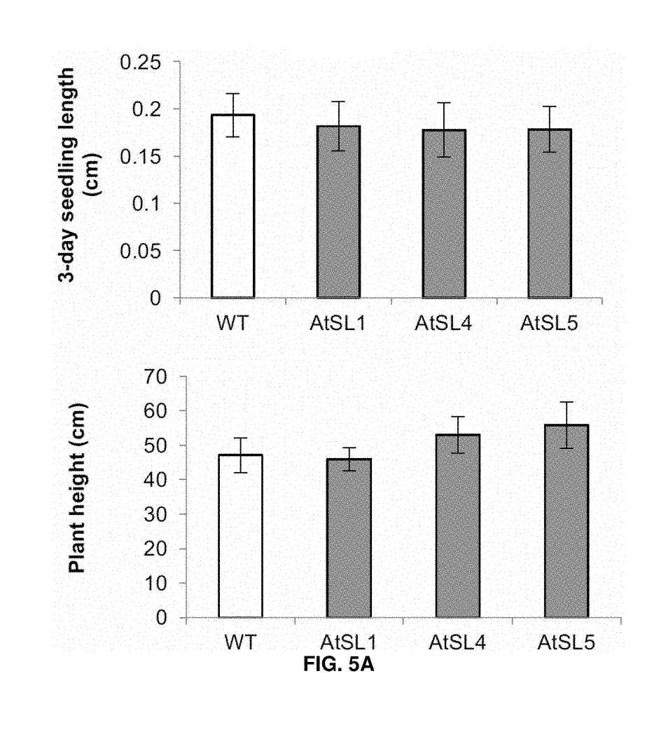

Twelve individual Arabidopsis plants of the wild type and each of the pSCPL17: ZmLEC1 transformed lines, and six individual Camelina plants of the wild type and each of the pACP5:ZmLEC1 and the pSCP17: ZmLEC1 transformed lines were grown under controlled conditions. Individual plants were arranged randomly in the trays to avoid edge effects that could bias the results. The selected AtSL4 and AtSL5 lines exhibited statistically significant (P<0.0001) increases in percentage seed oil content compared to the wild type (FIG. 4A). The seed oil content in AtSL5 reached 40%, which is an approximately 20% increase over the wild type. The selected CsAL5 and CsSL2 lines also exhibited statistically significant (P<0.001) increases in percentage seed oil content compared to the wild type. The 186 seed oil content in CsAL5 and CsSL2 was more than 40%, which is an approximately 16% increase over the content found in wild type seeds (FIG. 4B).

Overexpressing ZmLEC1 does not Adversely Affect Seed Vigor or Plant Growth

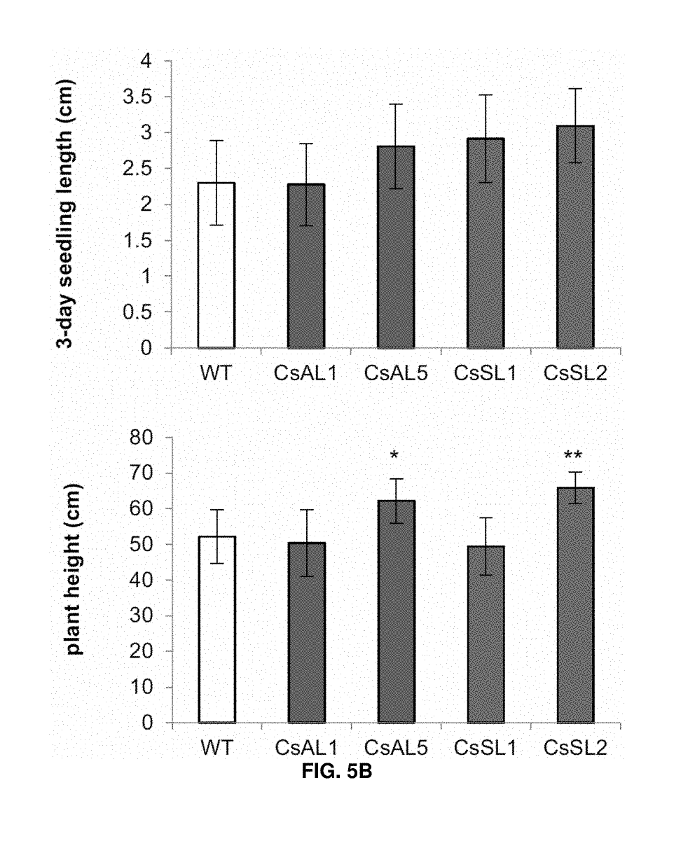

Considering the adverse growth effects reported in previous studies of overexpression of WRI1 and LEC1 [10, 19], it was important to assess the phenotype during the whole life cycle of the transgenic plants. We did not observe any obvious phenotypical differences in the transgenic plants and we specifically measured seedling and stem growth. Nearly all seeds germinated and seedling length three days post-germination showed no significant difference between the transgenic plants and the control plants (FIG. 5A). The transgenic plants showed a tendency to be taller than control plants, and this was significant in some of the Camelina lines (FIG. 5B).

### Overexpressing ZmLEC1 Plants Provide Increased Oil Yield

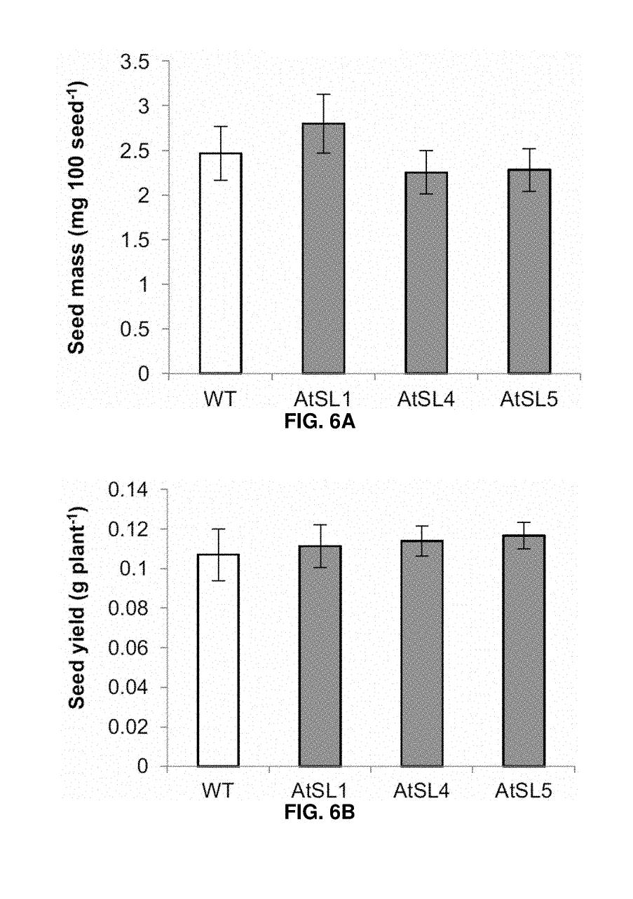

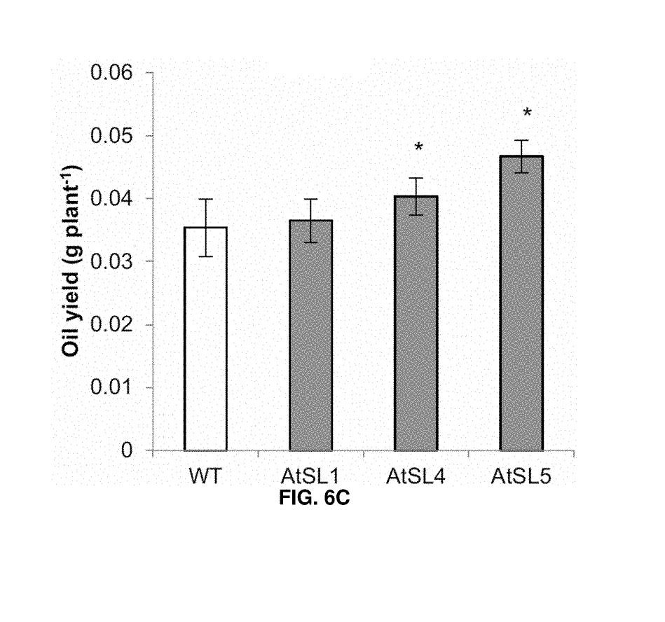

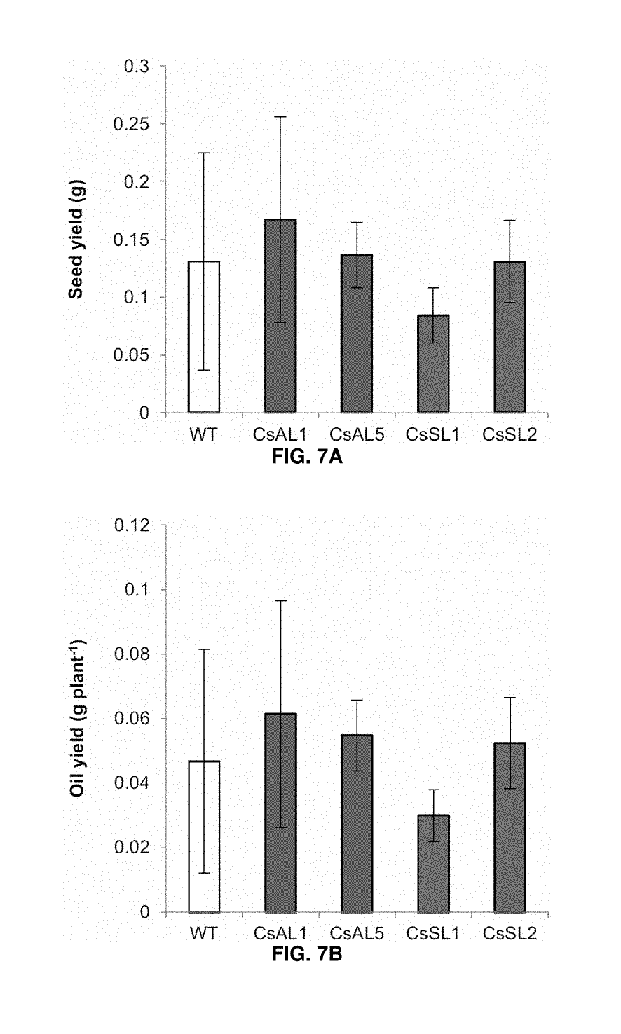

We examined additional traits of the transgenic lines to evaluate the total oil yield on a per plant basis as it depends on seed number per plant, seed size and oil fraction per seed. Analysis of 100-seed-weight showed that the expression of ZmLEC1, using down-stream promoters, does not result in a significant change in the average seed mass of Arabidopsis (FIG. 6A). In addition, all the seeds produced by each plant were collected to determine total seed yield in grams per plant (FIG. 6B, and FIG. 7A). There was a trend towards larger total seed yield in Arabidopsis while no significant change was observed in Camelina. From the total seed yield per plant (gram) and the seed oil content (percentage of seed weight), we calculated the total oil yield in grams per plant (FIGS. 6C and 7B). The data show that the total oil yield per plant is significantly (P<0.05) increased by 13% and 32% in the AtSL4 and AtSL5 lines, respectively (FIG. 6C). The total seed yield in the Camelina lines was relatively low probably because of suboptimal growth conditions for the plants. It will be important to repeat these studies in Camelina plants grown under optimal conditions and under field conditions.

### ZmLEC1 Overexpression Up-Regulates the Downstream Oil-Related Genes in Developing Seeds

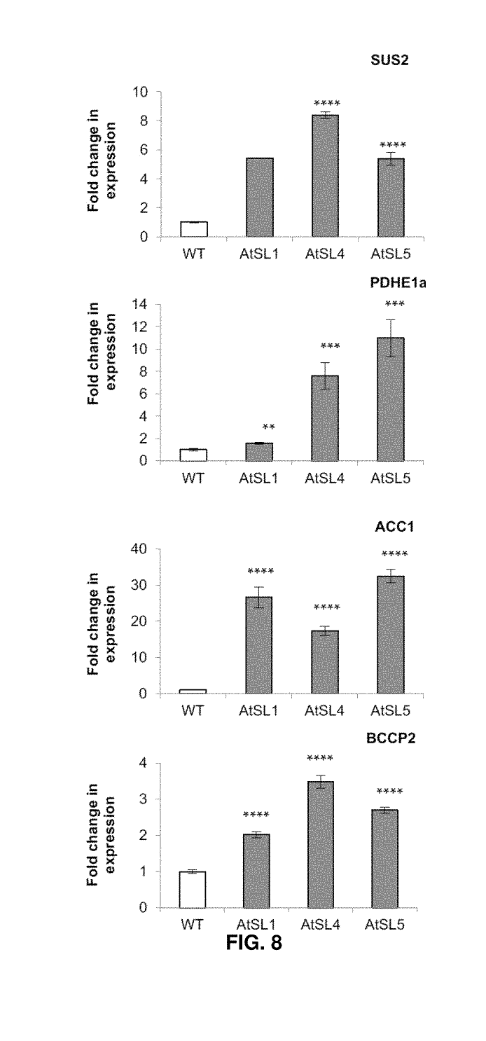

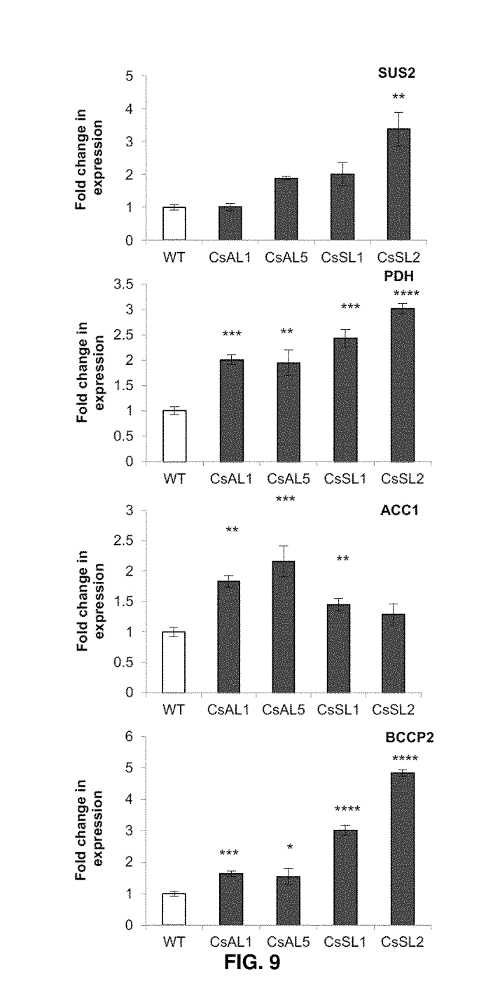

The transcript levels of a selection of oil-related genes were measured to examine the impact of the ZmLEC1 overexpression on these genes. In the transgenic Arabidopsis lines, several downstream genes regulated by AtLEC1 were up-regulated by about 2 to 10 fold compared with the wild-type plants: the sucrose synthase gene AtSUS2 (At5g49190), plastidic pyruvate dehydrogenase (PDH) E1a subunit (At1g01090) involved in late glycolysis, and acetyl CoA carboxylase (ACCase) BCCP2 subunit (At5g15530) involved in de novo fatty acid synthesis. The chain elongation related gene, ACC1 (At1g36160), was up-regulated 20˜30-fold (FIG. 8). In the transgenic Camelina lines, the corresponding CsSUS2 (XM_010441988), CsPDH (XM_010458872), CsACC1 (XM_010501769), and CsBCCP2 (XM_010455390) genes were all up-regulated by about 2 to 5-fold compared to those in wild-type plants (FIG. 9). In general, expression levels of the analyzed genes did not change as dramatically in Camelina as in Arabidopsis even though the increase in oil content was similar in the two species.

Experimental manipulation of ZmLEC1 caused transcriptional changes of genes coding for enzymes participating in sucrose metabolism, glycolysis, and FA biosynthesis, suggesting an enhanced carbon flux towards FA biosynthesis in tissues over-expressing ZmLEC1.

### DISCUSSION

Many scientists have recognized that overexpression of transcription factors provide an attractive solution for increasing plant oil production as compared to overexpression of pathway enzymes [14, 20, 21]. However, promoters must be chosen carefully to avoid negative effects while obtaining sufficient expression levels in target tissues. Van Erp and coworkers used a construct with a Sucrose Synthase 2 (Sus2) promoter to drive Arabidopsis WRI1 [14]. They obtained an approximately 10% increase in oil in Arabidopsis seeds with this promoter gene combination. An and Suh used the SiW6P promoter from Sesamum indicum (encoding linoleic acid desaturase) to drive the expression of the Arabidopsis WRI1 in Camelina [22]. They obtained seeds from 13 different transgenic lines, and the best had a 10.1% increase in seed fatty acid esters. A patent application described a variety of promoters used to drive WRI1 homologs from corn and other plants in Arabidopsis leading to 4.9% increase in oil with pSUC2:ZmODP (ODP is a WRI1 homolog) [23]. They also described the construct pSCP1:ZmODP, but oil was only increased by up to 2.6% in the best line. In our study, we tested several different seed-specific promoters and used them to drive the expression of ZmWRI1, AtWRI1, and ZmLEC1 in Arabidopsis and Camelina. The most promising preliminary results were obtained with ZmLEC1, which works upstream of WRI1. To identify the best promoters, we looked at the expression pattern using promoter-GUS constructs.

Based on the GUS analysis and comparison of different lines we could identify pSCPL17 (serine carboxypeptidase-like 17, At3g12203) as a strong and very seed-specific promoter. Another good promoter based on our analysis was pACP5 (Acyl Carrier Protein 5, At5g27200), which is stronger but less seed-specific. These promoter combinations with ZmLEC1 worked better than the previously published designs described above. In Arabidopsis lines transformed with pSCPL17:ZmLEC1 we find an increase in oil content of 21%, as compared to a maximum of 10% in the cited study [14].

In Camelina, a 16% increase in seed oil content was obtained with both pACP5:ZmLEC1 and pSCPL17:ZmLEC1 designs, as compared to the 10.1% obtained in the cited study [22]. With our transgene designs better results were obtained with ZmLEC1, as compared to those obtained with AtWRI1 or ZmWRI1, presumably because it works further upstream. Shen and coworkers obtained up to 30.6% increase in seed oil content in maize overexpressing WRI1 with seed-specific oleosin promoter and did not observe negative effects on growth or yield [10]. This interesting result is difficult to compare with results obtained with Arabidopsis and Camelina where seed oil content is naturally an order of magnitude higher than that observed in maize seeds and where such high oil yield increases have not previously been reported. Obviously, it will be interesting to test our promoter-transcription factor combinations in other crops such as canola, maize and soybean.

In general, it is important to choose 277 a promoter of the appropriate strength and specificity for constructs such as those described here. The ZmLEC1 overexpression can be combined with other engineering constructs, in particular, the overexpression of diacylglycerol acyl transferase (DGAT1) and downregulation of the lipase Sugar281 dependent 1 (SDP1) that were shown to increase the accumulation of oil when combined with overexpression of AtWRI1[14].

### CONCLUSIONS

Arabidopsis and Camelina lines over-expressing ZmLEC1 under the control of an Arabidopsis seed-specific promoter increased oil content in mature seeds by more than 20% in Arabidopsis and 16% in Camelina. Overexpression of ZmLEC1 does not appear to be detrimental to seed vigor under laboratory conditions. Furthermore, no abnormal growth phenotypes were observed throughout the life cycle of the plants. The findings demonstrated that a master regulator, ZmLEC1, driven by a downstream seed-specific promoter, can be used to influence oil production in Arabidopsis and Camelina and might be a promising transgene design for increasing oil production in various crops.

### Methods

### Plant Material and Growth Conditions

Wild-type (Col-0) Arabidopsis (Arabidopsis thaliana (L.) Heynh.) seeds were surface sterilized, sowed on agar plates containing one-half-strength Murashige and Skoog salts (Sigma-Aldrich) containing 1% sucrose, and incubated in darkness for 3 d at 4° C. The plates were then placed in a growth chamber set to 16 h/8 h day/night cycle, photosynthetic photon flux density 300 of 250 μmol m_{-2 s-1}, and 70% relative humidity. After 12 days, each seedling was transplanted to 7-cm₂ pots. Individual plants in each pot were arranged randomly in a tray. When plants began to flower, a 60-cm stick was inserted in each pot to tie the stems, and at maturity the pot was placed inside an upright rectangular transparent perforated glassine bag (60×6 cm). The bag was sealed around the lower stem prior to seed shedding to ensure that all the seeds from each plant were retained.

Wild-type Camelina (Camelina sativa (L.) Crantz, cultivar ‘Celine’) seeds were surface sterilized, sowed on agar plates containing one-half-strength Murashige and Skoog salts (Sigma-Aldrich) supplemented with 1% sucrose, and incubated in darkness for 3 d at 4° C.

The plates were then placed in a growth chamber set to 16 h/8 h day/night cycle, photosynthetic photon flux density of 250 μmol m_{-2 s-1}, and 70% relative humidity. After 5 days, each seedling was transplanted to 15-cm₂ pots. Individual plants in each pot were arranged randomly in a tray.

### Creation of DNA Constructs and Plant Transformation

A 1.9 kb promoter for AtSCPL17 (At3g12203; pSCP) and a 2.5 kb promoter for AtACP5 (At5g27200; pACP) were amplified by PCR from Arabidopsis genomic DNA using the SCP-F/R and ACP5-F/R primer pairs, respectively (Table 1), and cloned into pCR_Blunt (Thermo Fisher Scientific). The ZmLEC1 coding sequence was synthesized directly based on the amino acid sequence from maize (Zea mays leafy cotyledon 1, AF410176) without stop codon. The sequences corresponding to that of attb1 and attb2 were inserted during synthesis at the 5′-end and 3′-end of the ZmLEC1 coding sequence respectively.

TABLE 1
Details of primers used.

| | | |
|---|---|---|
| Primer name | Gene name | Primer sequence (5′-3′) |

Primer sequences for cloning

| | | |
|---|---|---|
| pSCP F | SCP17 | CCAAGCTTGGAAGAGCTCTTCTCTGGCTGTG |
| pSCP R | | GACCTAGGTCTCTTTGATCAAAAGTTTTT |
| pACP F | ACP5 | CCAAGCTTGCATACTCTCTCGTGAACTC |
| pACP R | | GACCTAGGTATCGATCTGATCGAGAG |
| ZmLEC1 F | LEC1 | GATGCCAAAGGGAGCAGAGA |
| ZmLEC1 R | | CCCCTTGCATCACCCTCAAA |
| F-Spect-pA6-SacII | SpecR | ccgattttgaaaccgcggCATGATATATCTCCCAATTTGTG |
| R-Spect-pA6-SacII | SpecR | ctgcctgtgatcaccgcggTAAGCCTCGTTCGGTTCGT |
| F-Basta-pA6-ApaI | BastaR | ctcggtaccaagcttggggcgcgccGATACATGAGAATTAAG |
| | | GGAGTC |
| R-Basta-pA6-AseI | BastaR | ctgaattaacgccgaattaatGAGCTTGCATGCCGGTCGATC |

Housekeeping gene primers in Arabidopsis

| | | |
|---|---|---|
| SAND F | SAND | AACTCTATGCAGCATTTGATCCACT |
| SAND R | | TGATTGCATATCTTTATCGCCATC |
| PP2AA3 F | PP2AA3 | TAACGTGGCCAAAATGATGC |
| PP2AA3 R | | GTTCTCCACAACCGCTTGGT |
| ER332 | | GAGCTGAAGTGGCTTCCATGAC |
| ER333 | | GGTCCGACATACCCATGATCC |

Primers for semi-quantitative/quanititative PCR of Arabidospsis

| | | |
|---|---|---|
| AtACC1 F | AtACC1 | AGTGAGAATGCATAGGTTGGG |
| AtACC1 R | | CTCGGTATATGTGGACAGTGC |
| AtBCCP2 F | AtBCCP2 | GACCCGGTGAACCCCCT |
| AtBCCP2 R | | GTCAACGCTGACTGGTTTTCCAT |
| AtPDHE1C F | AtPDHE1C | ATGTGTGCTCAAATGTATTACCGAGGC |
| AtPDHE1T R | | ACCTTTGCTGAGGGCATGG |
| AtSUS2 F | AtSUS2 | GCGGGAAGCAAGAACAATG |
| AtSUS2 R | | GAACAACTCGGTAAAGACCAGGC |

Housekeeping gene primers in Camelina

| | | |
|---|---|---|
| CsActin F | CsActin | ACA ATT TCC CGC TCT GCT GTT GTG |
| CsActin R | | AGG GTT TCT CTC TTC CAC ATG CCA |
| CsTubulin F | CsTubulin | GGGCTAAGGGACATTACACTG |
| CsTubulin R | | GTGTTCCCATACCAGATCCAG |
| CsEF1α F | CsEF1α | GGTAAGGAGATTGAGAAGGAGC |
| CsEF1α R | | CACAGCAAAACGTCCCAATG |

Primers for semi-quantitative/quantitative PCR of Camelina

| | | |
|---|---|---|
| CsACC1 F | CsACC1 | CTAAGCCCTGAAGACTACGAAC |
| CsACC1 R | | ATTACCCACCTTGTTTCCCC |
| CsBCCP2 F | CsBCCP2 | ACACAGTGGCATCTCCTTTC |
| CsBCCP2 R | | GGTTTTGGCTTTTCCGTTCAG |
| CsPDHE1T F | CsPDHE1T | TGGAGAATAACTTGTGGGCG |
| CsPDHE1A R | | ACCTTCAACACATCCATACCG |
| CsSUS2 F | CsSUS2 | CGTTTGCTACTTGTCATGGTG |
| CsSUS2 R | | TTACCCAGTGATTCGGATTGG |

The synthesized ZmLEC1 323 was then sub-cloned into the Gateway pDONR221-P1P2 (Zeocin) entry vector by BP recombination (Life Technologies). Two versions of vectors were generated for hygromycin and Basta selection of transformed Arabidopsis and Camelina lines, respectively. For the spectinomycin selection of bacteria, a spectinomycin marker was inserted in the backbone of the pA6-pC4H::GW vector [24] at the unique SacII restriction site. The spectinomycin marker was amplified from the pTKan vector [11] using the primer pair F-Spect-pA6-SacII/R331 Spect-pA6-SacII and inserted at the SacII restriction site using Gibson assembly method (New England Biolabs) to generate pA6Spect-pC4H::GW vector. The resulting vector was then digested by HindIII and AvrII to replace the pC4H promoter by pSCP and pACP to generate two new vectors: pA6Spect-pSCP::GW and pA6Spect-pACP::GW.

The Basta resistant vectors were generated by the replacement of the hygromycin marker in pA6Spect-pSCP::GW and pA6Spect-pACP::GW by that of BastaR. Both destination vectors were digested by ApaI and AseI to remove the hygromycin selection cassette. The BASTA marker was amplified by PCR using the primer pair F-Basta-pA6-ApaI/R339 Basta-pA6-AseI and pEARLYGATE [25] as template, then inserted between the ApaI and AseI to generate two destination vectors: pA6Spect-pSCP::GW-BASTA and pA6Spect-pACP::GW-BASTA.

The pA6Spect-pSCP::GUS and pA6Spect-pACP::GUS vectors were generated by LR cloning (Life Technologies) using pA6Spect-pSCP::GW and pA6Spect-pACP::GW destination vectors and a pDONR221-L1GUSL2 entry vector. Overexpression constructs containing the coding sequence 346 of ZmLEC1 fused to the Arabidopsis promoters were created the same way by LR cloning using pA6Spect-pSCP::GW, pA6Spect-pSCP::GW348 BASTA, pA6Spect-pACP::GW and pA6Spect-pACP::GW-BASTA destination vectors and the synthesized ZmLEC1 entry clone.

The recombination construct pA6Spect-XXX and pA6Spect-XXX-BASTA vectors were transformed into Agrobacterium tumefaciens strain GV3101 by heat shock and into Arabidopsis and Camelina, respectively, by the floral dip method [26]. Arabidopsis and Camelina transformants were selected on ½ MS medium supplemented with hygromycin (30 μg/mL) or Basta® (glufosinate ammonium; 30 μg/mL), respectively.

### Histochemical GUS Assay

For histochemical GUS staining, fresh samples from various tissues, including siliques, were incubated in X-Gluc solution [1 mM 5-bromo-4-chloro-3-indolyl-β-D-glucuronic acid, 50 mM phosphate buffer (pH 7.0), 2 mM potassium ferricyanide, 2 mM potassium ferrocyanide, 0.2% (v/v) Triton X-100] at 37° C. overnight. The staining buffer was then carefully removed, and the samples were washed three times (2-h washes) with ethanol:acetic acid (7:3) and stored in 95% ethanol.

### Genotyping

DNA extraction was following the manual of REDExtract-N-Amps Plant PCR Kits (Sigma-Aldrich), and PCR proceeded with leaf disk extract as template and ZmLEC1 F/R primer pairs. The PCR 368 condition was 30 cycles of denaturation at 94° C. for 30 s, annealing at 55° C. for 30 s, and elongation at 72° C. for 30 s.

### Transcript Analysis

DNase-treated total RNA was extracted from Arabidopsis siliques at 6 DAF, which corresponds to developmental stages 6 to 9 [18] and Camelina developing seeds at 15 DAF using RNeasy kit from Qiagen. The synthesis of single-stranded complementary DNA was carried out using iScript Reverse Transcription Supermix for RT-qPCR from BIO-RAD.

Primers were designed using the IDT DNA Real Time PCR primer design tool (webpage for: idtdna.com/scitools/Applications/RealTimePCR) (Table 1).

Quantitative real-time PCRs were performed on a STEPONE CFX96 Real-Time system (Applied Biosystems) and QuantiFast SYBR Green PCR kit from Qiagen following the manufacturers recommendation.

### Seed Oil Content Analysis

The percentage of oil in seeds was determined with a mini-spec mq10 nuclear magnetic resonance (NMR) analyzer (Bruker Optics Inc., Houston, Tex., USA). The mini-spec was calibrated by linear regression of NMR signals to weighed samples of pure Camelina oil following a general protocol provided by Bruker. Each seed sample was weighed and placed in the NMR tube and then measured against the calibration curve to determine the oil content. Calibration standards and seed samples were tempered at 40° C. for 0.5-1 hours before NMR measurement. The mini-spec 391 was operated at a resonance frequency of 9.95 MHz and was maintained at 40° C. Each measurement takes about 15 sec.

### Seed Germination and Seedling Growth Assays

The surface-sterilized seeds were plated on agar plates containing one-half-strength Murashige and Skoog salts (Sigma-Aldrich) containing 1% sucrose, and incubated in darkness for 3 d at 4° C. The plates were then placed vertically in a growth chamber set to 20° C. with constant low light (photosynthetic photon flux density of 10 μmol m_{-2 s-1}) for 3 days. Then the seedling length was measured.

### Statistical Analyses

The number of replicates (n) and the SE are shown for most measurements. One-way ANOVA was used to assess the differences between genotypes for measurements of seed percentage oil content, 3-day seedling length, plant height, seed mass, seed yield, oil yield and fold-change in gene expression. When the ANOVA showed significant differences (P<0.05) the individual lines were compared to wild type by Student's t-test, with significance level indicated in the figures.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of engineering a plant having an increased content of a target compound in the plant's seed, the method comprising introducing into the plant a first nucleic acid construct that encodes a seed-specific promoter operatively linked to a transcription factor wherein expression of the transcription factor increases the production of the target compound.

2. The method of claim 1, wherein the seed-specific promoter is serine carboxypeptidase-like (SCPL17) promoter or Acyl Carrier Protein (ACP5) promoter.

3. The method of claim 2, wherein the SCPL17 promoter is Arabidopsis thaliana SCPL17 promoter.

4. The method of claim 2, wherein the ACP5 promoter is Arabidopsis thaliana ACP5 promoter.

5. The method of claim 1, wherein the target compound is a lipid or fatty acid.

6. The method of claim 5, wherein the transcription factor is LEAFY COTYLEDON1 (LEC1) or WRINKLED 1 (WRI1).

7. The method of claim 4, wherein the LEC1 is Zea mays LEC1.

8. The method of claim 4, wherein the WRI1 is Zea mays WRI1 or Arabidopsis thaliana (WRI1).

9. The method of claim 1, wherein (i) the method further comprises introducing a second nucleic acid construct that encodes a seed-specific promoter operatively linked to a biosynthetic enzyme involved in the biosynthesis of the target compound, or (ii) the first nucleic acid construct further encodes a seed-specific promoter operatively linked to a biosynthetic enzyme involved in the biosynthesis of the target compound.

10. The method of claim 9, wherein the target compound is a lipid or fatty acid.

11. The method of claim 10, wherein the biosynthetic enzyme is diacylglycerol O-acyltransferase 1 (DGAT1).

12. The method of claim 1, wherein the method further comprises engineering the plant such that an endogenous enzyme, or an enzyme native to the plant, that decreases synthesis of, or catalyzes into another compound, the target compound is downregulated.

13. The method of claim 9, wherein the method further comprises engineering the plant such that an endogenous enzyme, or an enzyme native to the plant, that decreases synthesis of, or catalyzes into another compound, the target compound is downregulated.

14. The method of claim 12, wherein the target compound is a lipid or fatty acid.

15. The method of claim 14, wherein the enzyme is lipase sugar-dependent 1 (SDP1).

16. The method of claim 1, wherein the target compound is a protein, starch, or a storage polysaccharide, such as beta-glucan or mannan.

17. A genetically modified plant cell, comprising a first nucleic acid construct that encodes a seed-specific promoter operatively linked to a transcription factor wherein expression of the transcription factor increases the production of the target compound.

18. The genetically modified plant cell of claim 17, wherein the seed-specific promoter is serine carboxypeptidase-like (SCPL17) promoter or Acyl Carrier Protein (ACP5) promoter.

19. The genetically modified plant cell of claim 18, wherein the SCPL17 promoter is Arabidopsis thaliana SCPL17 promoter.

20. The genetically modified plant cell of claim 18, wherein the ACP5 promoter is Arabidopsis thaliana ACP5 promoter.

21. The genetically modified plant cell of claim 17, wherein the target compound is a lipid or fatty acid.

22. The genetically modified plant cell of claim 21, wherein the transcription factor is LEAFY COTYLEDON1 (LEC1) or WRINKLED 1 (WRI1).

23. The genetically modified plant cell of claim 22, wherein the LEC1 is Zea mays LEC1.

24. The genetically modified plant cell of claim 22, wherein the WRI1 is Zea mays WRI1 or Arabidopsis thaliana (WRI1).

25. The genetically modified plant cell of claim 17, further comprising a second nucleic acid construct that encodes a seed-specific promoter operatively linked to a biosynthetic enzyme involved in the biosynthesis of the target compound, or the first nucleic acid construct further encodes a seed-specific promoter operatively linked to a biosynthetic enzyme involved in the biosynthesis of the target compound.

26. The genetically modified plant cell of claim 25, wherein the target compound is a lipid or fatty acid.

27. The genetically modified plant cell of claim 26, wherein the biosynthetic enzyme is diacylglycerol O-acyltransferase 1 (DGAT1).

28. The genetically modified plant cell of claim 17, wherein the plant cell is engineered such that an endogenous enzyme, or an enzyme native to the plant cell, that decreases synthesis of, or catalyzes into another compound, the target compound is downregulated.

29. The genetically modified plant cell of claim 25, wherein the plant cell is engineered such that an endogenous enzyme, or an enzyme native to the plant cell, that decreases synthesis of, or catalyzes into another compound, the target compound is downregulated.

30. The genetically modified plant cell of claim 29, wherein the target compound is a lipid or fatty acid.

31. The genetically modified plant cell of claim 30, wherein the enzyme is lipase sugar-dependent 1 (SDP1).

32. The genetically modified plant cell of claim 17, wherein the target compound is a protein, starch, or a storage polysaccharide, such as beta-glucan or mannan.

33. A genetically modified plant or seed comprising a first nucleic acid construct that encodes a seed-specific promoter operatively linked to a transcription factor wherein expression of the transcription factor increases the production of the target compound.

34. A method of producing a target compound from a plant, comprising (a) optionally engineering a plant having an increased content of a target compound in the plant's seed using the method of claim 1, (b) growing the plant such that seeds are produced, (c) optionally harvesting the seeds produced by the plant, and (d) replanting the seeds harvested from step (c), or separating or isolating the target compound in the seeds harvested from step (c) from some, essentially all, or all of the other components of the seeds.
